(19) United States
(12) Patent Application Publication
(10) Pub. No.: US 2024/0109935 A1
(43) Pub. Date: Apr. 4, 2024

(54) PROCESS FOR THE PURIFICATION OF MONOCLONAL ANTIBODIES

(71) Applicants: COUNCIL OF SCIENTIFIC & INDUSTRIAL RESEARCH, New Delhi (IN); LUPIN LIMITED, Mumbai (IN)

(72) Inventors: Rahul Sharad BHAMBURE, Pune (IN); Prashant Sureshrao DEULGAONKAR, Pune (IN)

(21) Appl. No.: 18/276,567

(22) PCT Filed: Feb. 11, 2022
(86) PCT No.: PCT/IN2022/050121

§ 371 Date: Aug. 9, 2023

(30) Foreign Application Priority Data
Feb. 12, 2021 (IN) 202111006130

(51) Int. Cl. C07K 1/36; C07K 1/18; C07K 1/22; C07K 1/34; C07K 16/28

(52) U.S. Cl. CPC C07K 1/36; C07K 1/18; C07K 1/22; C07K 1/34; C07K 16/2803; C07K 2317/14

(57) ABSTRACT

The present invention relates to a process for purifying a protein of interest in a batch, integrated continuous or pseudo-continuous mode. Accordingly, the present invention relates to a process for purifying monoclonal antibodies, specifically IgG. in a batch, integrated continuous or pseudo-continuous mode. The process can be executed in batch, continuous, integrated continuous or pseudo-continuous modes.

### FIELD OF INVENTION

The present invention relates to a process for purifying a protein of interest in a batch, integrated continuous or pseudo-continuous mode. Accordingly, the present invention relates to a process for purifying monoclonal antibodies, specifically IgG in a batch, integrated continuous or pseudo-continuous mode.

### BACKGROUND OF INVENTION

For recombinant biopharmaceutical proteins to be acceptable for administration to human patients, residual impurities resulting from the manufacture and purification process must be removed from the final biological product. These process components include culture medium proteins, immunoglobulin affinity ligands, viruses, endotoxin, DNA, and host cell proteins (HCPs).

With increasing cell culture titers and larger cell culture volumes being used for production, downstream processing is viewed as a bottleneck in industry. This is particularly relevant to monoclonal antibody (mAb) production, where the focus has shifted away from batch volume towards downstream processing capacity.

Downstream processing is currently faced with several barriers that may limit its ability to process large monoclonal antibody (mAb) batch sizes as a result of an increase in upstream productivity. One of the challenges is the limitation in space and operation flexibility in existing manufacturing facilities. To recover the increased mass of product from upstream feedstock, the chromatography column needs to be larger, thus the amount of resin needed, filter surface area and buffer and intermediate pool volumes also increase (Low, 2007). With the increase in process intermediate volumes, the existing pool tanks are often too small to accommodate the larger pool volume, and therefore becomes the bottleneck in large-scale production of high titer processes.

Single Pass Tangential Flow Filtration (SPTFF) is a new technology that aims to concentrate protein solutions in a single pass, without the need for a recirculation loop and tank (De Los Reyes G, 2008). The novelty in the design of the SPTFF module is the arrangement of multiple filtration stages in a single unit to concentrate proteins in a single passage, thus removing the need for a recirculation tank required with traditional TFF operations. With the SPTFF modules, adding more levels of membranes increases the path length and the residence time of the solution in the filter, thus a larger amount of permeate is removed and the feed volume decreases with just one pass. Due to its single-pass mode, the SPTFF feedstock remains at a constant protein concentration, which allows the system to come to steady-state equilibrium. Therefore, with a constant feed flow rate (Q feed), the retentate flow rate (Qretentate), filtrate flow rate (Qfiltrate), and concentration factor also remain constant during the SPTFF operation.

Although remarkable progress has been achieved in this research area in the past, that uses advanced filtration techniques including SPTFF.

US Patent Publication No. 20150361129 describes the process for separating target proteins from non-target proteins. The sample containing the target and non-target proteins was concentrated by single-pass tangential flow filtration (SPTFF). Further purification of target proteins was performed in batch mode. There is a need to perform a continuous capture chromatography step after concentrating the target and non-target proteins.

U.S. Pat. No. 5,429,746 discloses a method for separating IgG monomers from aggregates in mixtures comprising the steps of Protein A, ion-exchange chromatography, hydrophobic interaction chromatography, and filtration techniques including tangential-flow ultra filtration.

US Patent Publication No. 20100234577 discloses the purification of monoclonal antibodies from mammalian cell culture fluid utilizing sequential, orthogonal chromatography, and filtration techniques including tangential flow ultra filtration (TFUF), resulting in the material of high purity and quality suitable for human administration.

US Patent Publication No. 20180155752 describes a method for production and purification of cell products including immunoglobulins by performing the steps of affinity chromatography, immuno-affinity chromatography, ionic exchange chromatography, hydrophobic interaction chromatography, tangential flow filtration, and size selection chromatography (SEC).

On analyzing the literature pertaining to protein purification procedures, there appears a need in the art to provide a purification process that considers the drawbacks posed by the purification procedures of the prior art disclosures.

### OBJECT OF INVENTION

Accordingly, the main objective of the present invention is to provide a process for purifying proteins in a batch, integrated-continuous or pseudo-continuous mode.

Further, another object of the present invention is to provide a process for purifying IgG antibodies in a batch, integrated continuous or pseudo-continuous mode.

### SUMMARY OF THE INVENTION

Accordingly, the present invention provides a process for purifying a protein of interest in batch, integrated continuous or pseudo-continuous mode.

In an embodiment of the present invention, there is provided a process for purifying a protein of interest in batch, integrated continuous or pseudo-continuous mode, comprising the steps of,
(a) filtering a harvest material containing monoclonal antibody using an AEX hybrid filter (anion exchange functionality integrated with membrane) to clarify harvest and remove process-related impurities generated during upstream processing selected from the group consisting of turbidity, HCP, and DNA;
(b) concentrating the AEX hybrid filter output using a first single-pass tangential flow filtration (SPTFF) system or a first in-line concentrator (ILC) to obtain a concentrated monoclonal antibody solution prior to capture step purification;
(c) subjecting the concentrated monoclonal antibody solution from step (b) to a Protein A affinity chromatography, wherein the said chromatography process comprising;
   (i) loading the concentrated monoclonal antibody solution of step (b) at pH range from 6.00 to 9.50 with conductivity more than 1 mS/cm to an affinity chromatography resin to capture monoclonal antibody and remove process and product-related impurities,
   (ii) eluting the captured monoclonal antibody using an elution buffer in pH range from 2.00 to 5.00 with conductivity more than 0.5 mS/cm,
      wherein loading on the affinity chromatography resin in step, (i) is performed at >0.1% of breakthrough capacity,
      wherein the number of chromatography columns used in step (i) of affinity chromatography purification is at least one,
   wherein elution in affinity chromatography is performed using pH range from 2.00 to 5.00 or a salt-based gradient;
(d) Subjecting a protein A elution from step (c) to viral inactivation at pH range from 2.00 to 4.00 with conductivity more than 0.5 mS/cm in a batch, a pseudo-continuous mode, or a continuous mode;
(e) Subjecting the post-viral inactivated liquid output obtained in step (d) to a cation exchange or an anion exchange chromatography for separation of process and product-related impurities;
   wherein, the anion exchange chromatography is performed in a flow-through mode for separation of process-related impurities at pH 6.50 to 8.50 with conductivity more than 0.5 mS/cm,
   wherein the cation exchange chromatography is performed in a bind and elute mode for separation of product-related impurities at pH to 4.00 to 7.00 with conductivity more than 0.5 mS/cm;
(f) concentrating the cation exchange chromatography or Anion exchange chromatography output using a second single-pass tangential flow filtration (SPTFF II) system or a second in-line concentrator (ILC II) to form a retentate; and
(g) subjecting the retentate of step (f) for a viral filtration to produce purified monoclonal antibody.

In an embodiment of the present invention, the selected mAb product is produced using a batch, a fed-batch or a continuous cell culture process.

In another embodiment of the present invention, the selected mAb product is produced in a continuous cell culture process.

In still another embodiment of the present invention, the harvest material is a CHO harvest.

In a preferred embodiment of the present invention, the continuous downstream process is a part of a manufacturing process that exhibits a 5× increase in productivity over a batch process (where x is the productivity of batch process).

In another embodiment of the present invention, the protein of interest is a monoclonal antibody.

In a preferred embodiment of the present invention, the monoclonal antibody is an IgG.

### DETAILED DESCRIPTION OF THE DRAWINGS

The invention has other advantages and features which will be more readily apparent from the following detailed description of the invention and the appended claims, when taken in conjunction with the accompanying drawings, in which:

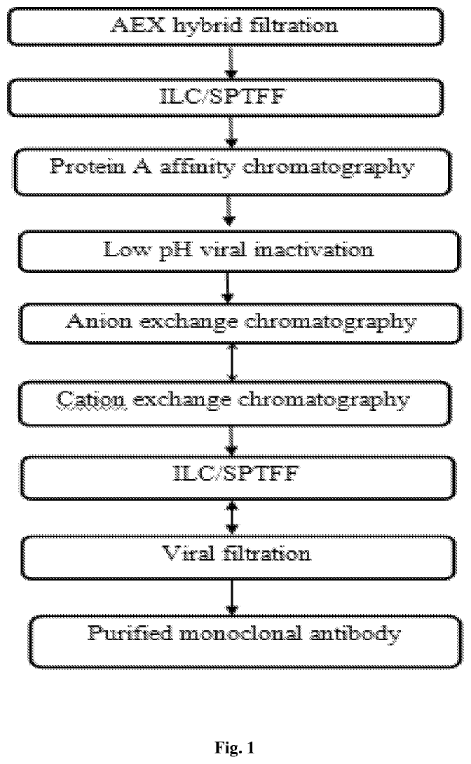

FIG. 1: depict the method for purifying monoclonal antibody specifically IgG in batch, integrated continuous or pseudo-continuous mode.

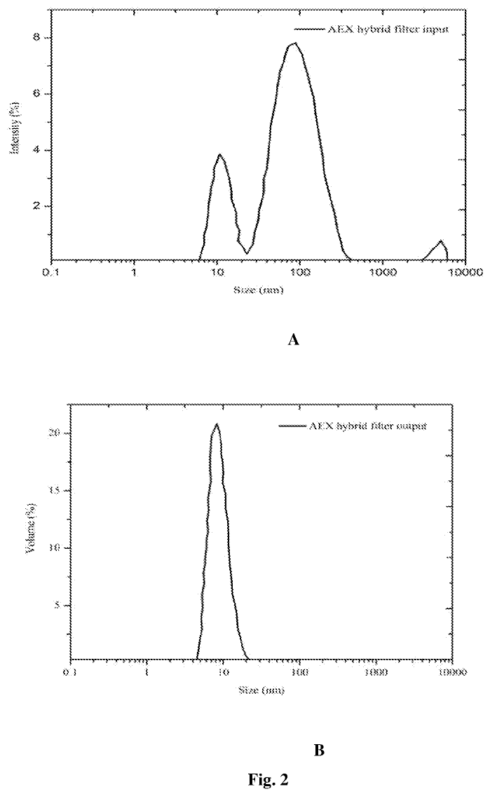

FIG. 2: depicts the particle size distribution measured for A] CHO harvest containing mAb solution before AEX filtration, B] AEX filtration output.

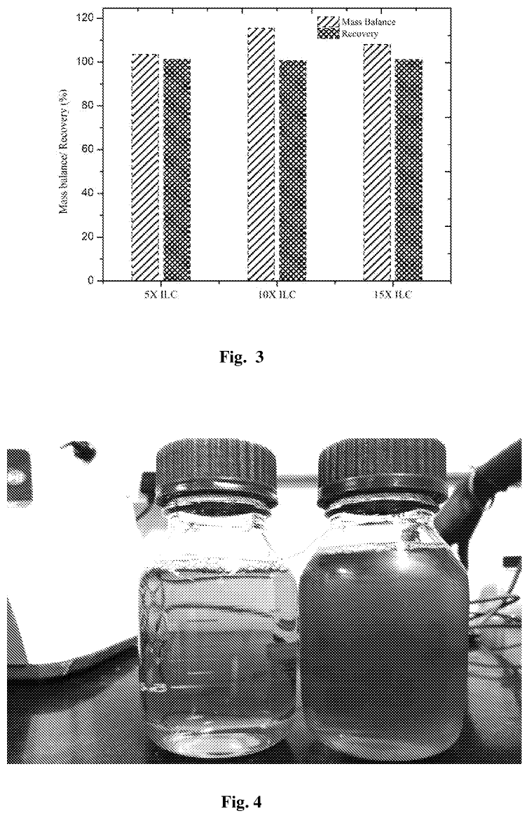

FIG. 3: depicts the mass balance and recovery values for 5×, 10× and 15× concentration experiments performed using ILC/SPTFF.

FIG. 4: depicts the turbid metric change associated with the concentration effect obtained using ILC or SPTFF.

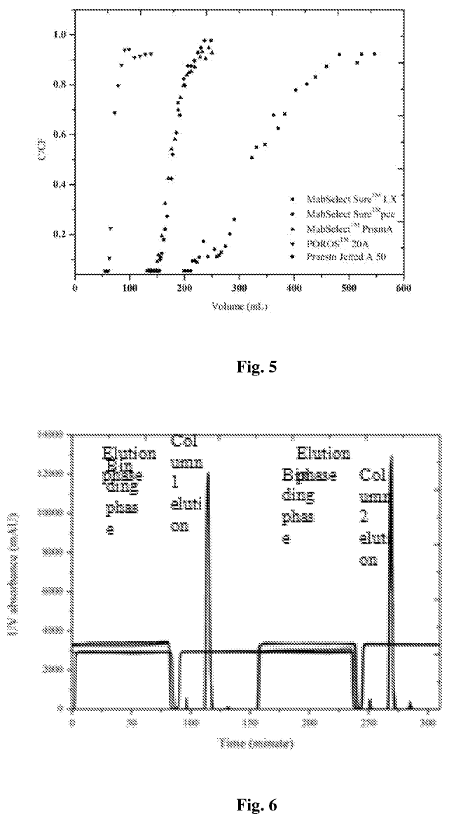

FIG. 5: depicts the breakthrough curve for MabSelect SuRe™ LX, MabSelect SuRe™ pcc, MabSelect™ PrismA, POROS® 20A, Praesto® Jetted A50 protein A affinity resin at 6 minute residence time.

FIG. 6: depicts the reproducibility of 3 cycles run for continuous capture of antibody solution.

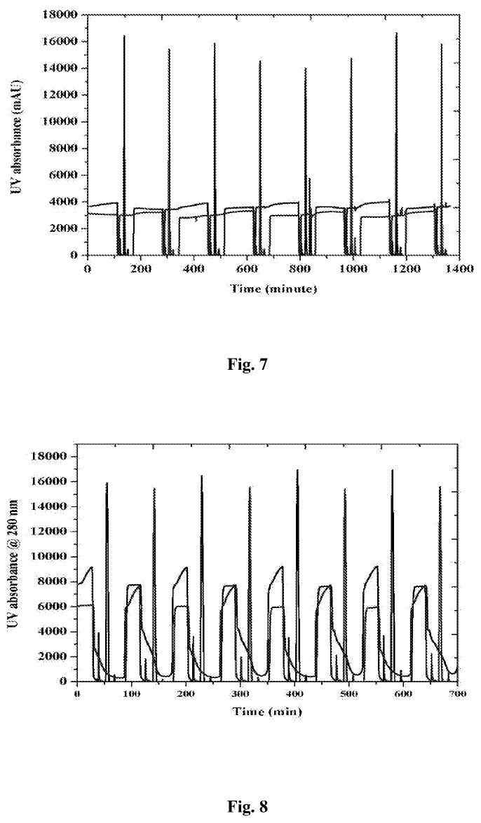

FIG. 7: depicts the four cycle continuous protein A chromatography experiment for 1× feed concentration.

FIG. 8: depicts the four cycle continuous protein A chromatography experiment for 5× feed concentration.

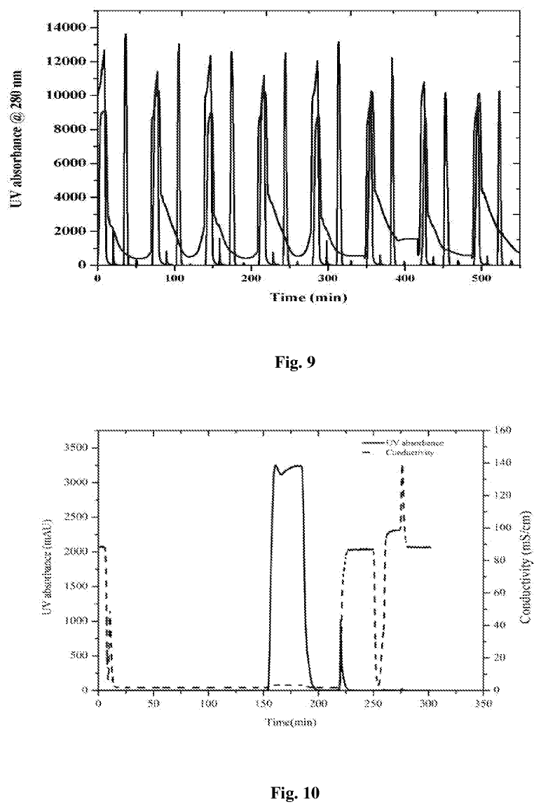

FIG. 9: depicts the four cycle continuous protein A chromatography experiment for 15× feed concentration.

FIG. 10: depicts the chromatogram of anion exchange experiment on Q Sepharose® Fast Flow.

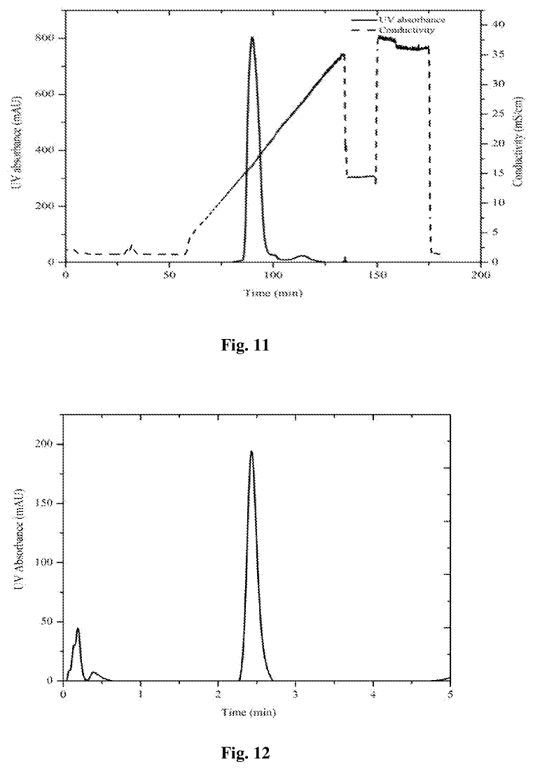

FIG. 11: depicts the chromatogram for cation exchange chromatography using Fractogel® SO₃⁻ (M) resin.

FIG. 12: depicts the analytical Protein A chromatogram for quantitative estimation of monoclonal antibody product in various process outputs.

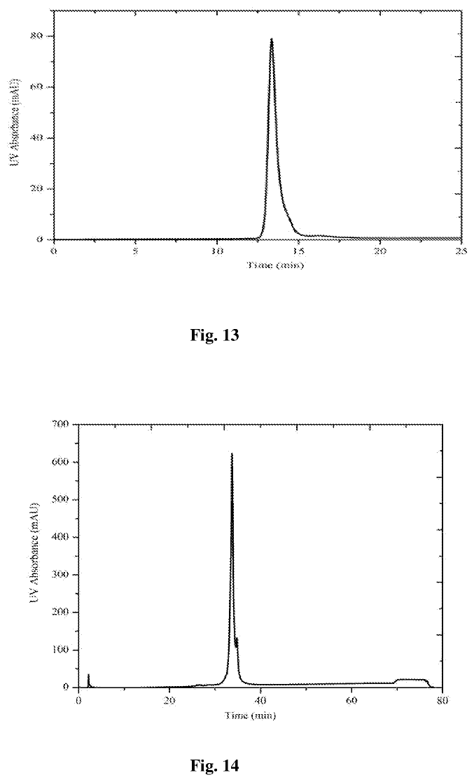

FIG. 13: depicts the analytical size exclusion chromatogram for aggregation and fragments analysis of monoclonal antibody product.

FIG. 14: depicts the analytical cation exchange chromatogram for charge variant analysis of monoclonal antibody product.

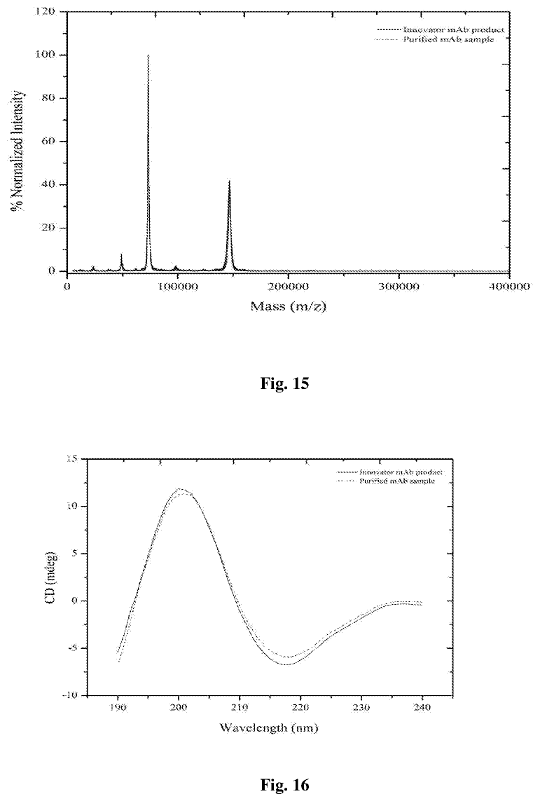

FIG. 15: depicts the intact mass analysis of purified monoclonal antibody using MALDI-TOF analysis.

FIG. 16: depicts the far CD spectroscopic analysis of monoclonal antibody purified using the developed purification process.

### DETAILED DESCRIPTION OF THE INVENTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The tables, figures and protocols have been represented where appropriate by conventional representations in the drawings, showing only those specific details that are pertinent to understanding the embodiments of the present invention so as not to obscure the disclosure with details that will be readily apparent to those of ordinary skill in the art having benefit of the description herein.

As used herein, the terms “VCF value” when used in the context of the present invention refers to volumetric concentration factor is the amount that the feed stream has been reduced in volume from the initial volume.

Accordingly, to accomplish the objectives of the present invention, the inventors propose a method for purifying a protein of interest in a batch, integrated-continuous or pseudo-continuous mode.

In an embodiment of the present invention, there is provided a process for purifying a protein of interest in a batch, an integrated continuous or a pseudo-continuous mode, which is depicted in FIG. 1, comprising the steps of,
(a) filtering a harvest material containing monoclonal antibody using an AEX hybrid filter (anion exchange functionality integrated with membrane) to clarify harvest and remove process-related impurities generated during upstream processing selected from the group consisting of turbidity, HCP, and DNA;
(b) concentrating the AEX hybrid filter output using a first single-pass tangential flow filtration (SPTFF) system or a first in-line concentrator (ILC) to obtain a concentrated monoclonal antibody solution;

(c) subjecting the concentrated monoclonal antibody solution from step (b) to a Protein A affinity chromatography, wherein said chromatography process comprising,
(i) loading the concentrated monoclonal antibody solution of step (b) at pH range from 6.00 to 9.50 with conductivity more than 1 mS/cm to an affinity chromatography resin to capture monoclonal antibody and remove process and product-related impurities,
(ii) eluting the captured monoclonal antibody using an elution buffer in pH range from 2.00 to 5.00 with conductivity more than 0.5 mS/cm,
   wherein loading on the affinity chromatography resin in step (i) is performed at >0.1% of breakthrough capacity,
   wherein the number of chromatography columns used in step (i) of affinity chromatography purification is at least one,
   wherein elution in affinity chromatography is performed using pH range from 2.00 to 5.00 or a salt-based gradient,
(d) subjecting a protein A elution from step (c) for viral inactivation at pH range from 2.00 to 4.00 with a conductivity more than 0.5 mS/cm in a batch, a pseudo-continuous mode, or a continuous mode;
(e) subjecting the post-viral inactivated liquid output obtained in step (d) to a cation exchange or an anion exchange chromatography for separation of process and product-related impurities,
   wherein the anion exchange chromatography is performed in a flow-through mode for separation of process-related impurities at pH range from 6.50 to 8.50 with conductivity more than 0.5 mS/cm,
   wherein the cation exchange chromatography is performed in a bind and elutes mode for separation of s product-related impurities at pH range from 4.00 to 7.00 with conductivity more than 0.5 mS/cm,
(f) concentrating the cation exchange chromatography or Anion exchange chromatography output of step (e) using a second single-pass tangential flow filtration (SPTFF II) system or a second in-line concentrator (ILC II) to obtain a retentate; and
(g) Subjecting the retentate of step (f) for viral filtration to produce the purified monoclonal antibody.

In still another embodiment of the present invention, the harvest material is a CHO harvest.

In another embodiment of the present invention, the protein of interest is a monoclonal antibody.

In still another embodiment of the present invention, the process for purifying monoclonal antibody in a batch, an integrated continuous or a pseudo-continuous mode comprises an AEX-filtration step performed at pH range from 6.00 to 9.50 with conductivity more than 0.5 mS/cm to affinity chromatography.

In yet another embodiment of the present invention, when the AEX-hybrid filtration unit is more than 1, they are connected in parallel or in series with a suitable pressure or time or volume or protein concentration based flow and flow path controller to facilitate the AEX hybrid filtration operation in a continuous mode.

In a further embodiment of the present invention, the HCP and HC-DNA log reduction achieved using the AEX-filtration step is not less than 0.5.

In still another embodiment of the present invention, the turbidity of the AEX-filtration output is not more than 10.0 NTU.

In yet another embodiment of the present invention, when the single-pass tangential flow filtration (SPTFF) or in-line concentrator (ILC) are more than 1, they are connected in-parallel or in-series with a suitable pressure or time or volume or protein concentration based flow and flow path controller to facilitate the SPTFF/ILC operation in a continuous mode.

In a further embodiment of the present invention, the single pass tangential flow filtration (SPTFF) or in-line concentrator (ILC) is performed at pH range from 6.00 to 9.50 with conductivity more than 0.5 mS/cm to an affinity chromatography.

In an embodiment of the present invention, the volumetric concentration factor (VCF) achieved using single pass tangential flow filtration (SPTFF) or in-line concentrator (ILC) units is not less than 1.1× where X is the initial protein concentration of the feed. The VCF values for 5×, 10× and 15× concentration experiments were determined using ILC/SPTFF and were found to be not less than 1.1×.

In another embodiment of the present invention, the AEX-hybrid filtration and SPTFF or ILC units are used single or multiple times.

In still another embodiment of the present invention, the monoclonal antibody concentration in ILC/SPTFF output is always higher as compared to monoclonal antibody concentration in harvest.

In yet another embodiment of the present invention, a feed inlet of the SPTFF or ILC system is connected to an outlet of the AEX hybrid filter and a retentate outlet of SPTFF or ILC system is connected to the protein A affinity chromatography.

In a preferred embodiment of the present invention, residence time for Protein A based affinity chromatography, anion exchange chromatography and cation exchange chromatography is more than 15 second.

In yet another embodiment of the present invention, the Protein A based affinity chromatography, anion exchange chromatography or cation exchange is performed using an axial or a radial flow chromatography column.

In still another embodiment of the present invention, the Protein A based affinity chromatography is performed using a Protein A ligand immobilized on a natural polymer bead based or a synthetic polymer bead based, a membrane based, a hydrogel based or a fibre based chromatography matrix.

In further embodiment of the present invention, the Protein A based affinity chromatography matrix is selected from the group consisting of MabSelect SuRe™, MabSelect SuRe™ LX, MabSelect SuRe™ pcc, MabSelect™ PrismA, Fibro PrismA, Praesto® Jetted A50, Amsphere™ A3, TOYOPEARL®, AF-rProtein A HC-650F, KANEKA KanCapA™ KANEKA KanCapA™ 3G, Eshmuno® A, Praesto® AP, and MabSpeed™ rP202.

In an embodiment of the present invention, the anion exchange chromatography is performed using an anion exchange functional groups selected from the group consisting of diethylaminoethyl, quaternary ammonium, and polyethyleneimine, trimethylammoniumethyl, linked to a bead based, a membrane based, a hydrogel based or a fibre based chromatography matrix.

In a preferred embodiment of the present invention, the anion exchange chromatography comprises a resin or a membrane selected from the group consisting DEAE Sepharose® Fast Flow, Q Sepharose® Fast Flow, SOURCE™ 15Q, SOURCE™ 30Q, Fractogel® EMD DEAE, POROS® 50 HQ, Nuvia™ Q, Capto™ ImpRes Q. Capto™ Q, Capto™ DEAE, Fractogel® EMD TMAE, Fractogel® EMD DMAE, Natrix® Q, Sartobind® Q, and Mustang® Q.

In still another embodiment of the present invention, the total amount of monoclonal antibody product passing through the single anion exchange chromatography column is not less than 30 g/L resin.

In a yet another embodiment of the present invention, the cation exchange chromatography is performed using a cation exchange functional groups selected from the group consisting of a sulfonate group, a sulfopropyl group and a sulphonic acid linked to a bead based, a membrane based, a hydrogel based and a fibre based chromatography matrix.

In preferred embodiment of the present invention, the cation exchange chromatography comprises a resin or a membrane selected from the group consisting of Fractogel® EMD SO₃⁻ (M), Capto™ SP ImpRes, POROS® XS, Nuvia™ S, SOURCE™ 15S, SOURCE™ 30S, SP Sepharose® Fast Flow, Natrix® HD-Sb, Sartobind® S, and Mustang® S.

In yet another embodiment of the present invention, the gradient elution in cation exchange chromatography is achieved using a salt gradient and or a pH gradient.

In an embodiment of the present invention, the cation exchange chromatography column has a dynamic binding capacity for the monoclonal antibody of more than 10 g/L resin.

In another embodiment of the present invention, the purified antibody drug substance contains no more than 0.3% soluble aggregate.

In a preferred embodiment of the present invention, the purified antibody drug substance contains no more than 100 ppm HCP in final drug substance.

In still another embodiment of the present invention, the purified antibody drug substance contains no more than 10 ng/mL of DNA in final drug substance

In an embodiment of the present invention, the selected mAb product is produced using a batch, a fed-batch or a continuous cell culture process.

In another embodiment of the present invention, the selected mAb product is produced in a continuous cell culture process.

In a preferred embodiment of the present invention, the selected mAb product is an IgG.

In a further embodiment of the present invention, the continuous downstream process is a part of a manufacturing process that exhibits a 5× increase in productivity over a batch process (where x is the productivity of batch process).

In a preferred embodiment of the present invention, the process produces a protein yield of 30 g/L/h.

In yet another embodiment of the present invention, the effect of VCF enhancement till 15× can be made 25× to achieve the higher production values.

In yet one more embodiment of the invention, the mass balance of the process is >100% and yield of the purified mAb is >90% and is independent of the initial concentration.

### EXAMPLES

The following examples, which include preferred embodiments, will serve to illustrate the practice of this invention, it being understood that the particulars shown are by way of example and for purpose of illustrative discussion of preferred embodiments of the invention.

### Example 1: AEX Hybrid Filtration

Filtration of harvest material containing monoclonal antibody was performed using AEX filter (anion exchange functionality integrated with membrane) to clarify harvest and remove process-related impurities generated during upstream processing (Turbidity, HCP, and DNA). 20.0 mM sodium phosphate buffer containing 150.0 mM NaCl at pH 7.40±0.20 was used for filter equilibration, Harvest containing IgG is filtered through AEX (anion exchange functionality integrated with membrane) hybrid filter at a flux of 222.0 LMH with a throughput of 250.0 L/m2. Table 1 list the values of host cell protein and DNA impurities obtained using AEX hybrid filtration of the harvest. FIG. 2 indicates the removal of chromatin impurity using AEX-hybrid filtration using dynamic light scattering measurement.

TABLE 1
Host cell protein and DNA impurity level in AEX filter output

| | | | | HC- | |
|---|---|---|---|---|---|
| | | HCDNA | HCP | DNA log | HCP log |
| Sample | Turbidity | (ng/ml) | (ng/ml) | reduction | reduction |
| AEX- | 1.88 ± | 697.54 | 373986.26 | 1.02 | 0.09 |
| Hybrid | 0.5 | | | | |
| filter | | | | | |
| output | | | | | |

### Example 2: Single Pass Tangential Flow Filtration or In-Line Concentration

The in-line concentration of AEX-filer output containing monoclonal antibody solution was performed using Cadence™ in-line concentrator. The desired concentration of the mAb (5×, 10×, or 15×) was achieved by adjusting feed pressure and the inlet flow rate of the harvest. The operation was performed at 32 LMH flux with a throughput of 272.32 L/m2. 20.0 mM sodium phosphate buffer containing 150.0 mM NaCl at pH 7.40±0.20 was used as an equilibration buffer for the single-pass tangential flow filtration operation. FIG. 3 indicate the mass balance and recovery of the mAb obtained in 5×, 10×, and 15× concentration experiment. FIG. 4 indicates the turbidimetric change associated post concentration of the AEX-filter output using the ILC/SSPTFF.

### Example 3: Breakthrough Curve Measurement for Various Protein a Affinity Resins Experiment

To measure the breakthrough curve, Protein A affinity resin is overloaded until monoclonal antibody solution concentration in column output matches with the initial mAb concentration in feed. The affinity resin was equilibrated using 20 mM Sodium phosphate containing 150 mM NaCl, pH 7.40±0.20 buffer. The concentrated monoclonal antibody solution (In-line concentrator output) was loaded on Protein A affinity resin at two minute residence time. Post loading column is washed and eluted with 20 mM sodium phosphate buffer containing 150 mM NaCl, pH 7.40±0.20 and 100 mM Glycine HCl, pH 3.00±0.20 respectively. The breakthrough curves for MabSelect SuRe™ LX protein A affinity resin, MabSelect SuRe™ PCC protein A affinity resin, MabSelect SuRe™ PrismA protein A affinity resin, Praesto® Jetted A50 protein A affinity resin, POROS™ 20 A protein A at 6 minute residence time was determined at a 6 minute residence time.

### Example 4: Twin Column Protein Affinity Chromatography for Capture Step Purification of mAb

Protein A affinity chromatography was performed using MabSelect™ PrismA resin. Affinity chromatography was performed on a twin column chromatography system using Contichrom® CUBE+ from ChromaCon AG. The concentrated monoclonal antibody solution (SPTFF/ILC output) was used as feed material. Experiment to capture IgG from concentrated material was conducted using 20 mM Sodium phosphate containing 150 mM NaCl, pH 7.40±0.20 as an equilibration buffer, 20 mM Sodium phosphate containing 500 mM NaCl, pH 7.40±0.20 as a wash buffer, 100 mM Glycine HCl, pH 3.00±0.20 as an elution buffer, 100 mM Glycine HCl, pH 2.00±0.20 as a strip buffer and 0.1 M NaOH was used for cleaning of resin. Elution was performed using step pH gradient. All the process steps were performed at two-minute residence time. FIG. 5 shows a breakthrough curve for MabSelect SuRe™ LX, MabSelect SuRe™ pcc, MabSelect™ PrismA, POROS® 20A, Praesto® Jetted A50 protein A affinity resin at 6 minute residence time.

To enhance the utilization of Protein A affinity resin, capacity loading at various breakthrough curve % was done. Table 2 shows the impact of different breakthrough curve 20% on productivity; recovery and mass balance of Protein A affinity capture step. It was proved that productivity increases with an increase in breakthrough curve %. FIG. 6 depicts the reproducibility of 3 cycles run for continuous capture of antibody solution.

TABLE 2
Impact of different breakthrough curve % loading on
productivity, recovery and mass balance of capture step

| Parameter | Unit | 60% of BTC | 70% BTC | 80% BTC |
|---|---|---|---|---|
| Yield | [%] | 95.2 | 104.9 | 104.9 |
| Mass balance | [%] | 96.2 | 104.9 | 106.3 |
| Productivity | [g/L/h] | 11.87 | 11.53 | 12.39 |

Impact of Cell Culture Titer on Productivity of Capture Process:

FIG. 7 to FIG. 9 shows continuous capture run chromatograms at 1×, 5×, 15× feed titer obtained using the ILC/SPTFF. Productivity of continuous capture process increases with increase in feed titer as shown in table 3. Table 4 shows the impurity content in SPTFF/ILC and Protein A affinity chromatography output.

TABLE 3
Impact of feed titer on productivity, recovery and
mass balance of capture step

| Parameter | Unit | Without ILC | 5X | 10X | 15X |
|---|---|---|---|---|---|
| Yield | [%] | 104.9 | 93.3 | 104.3 | 93.7 |
| Mass balance | [%] | 106.3 | 105.8 | 105.8 | 94.1 |
| Productivity | [g/L/h] | 12.39 | 20.07 | 26.65 | 25.59 |

TABLE 4
Impurity content in In-Line concentration and affinity
chromatography output

| | HCDNA | HCP | HC-DNA log | HCP log |
|---|---|---|---|---|
| Sample | (ng/mL) | (ppm) | reduction | reduction |
| In-line concentration | 10723.97 | 150633.95 | Not | 0.46 |
| (ILC) output | | | applicable | |
| Affinity | 393.79 | 142.47 | 1.44 | 3.02 |
| chromatography | | | | |
| output | | | | |

### Example 5: Anion Exchange Chromatography for Separation of Various Process Related Impurities

Anion exchange chromatography was performed using DEAE Sepharose® Fast Flow and Q Sepharose® Fast Flow resin as shown in chromatogram FIG. 10. The output monoclonal antibody solution of Protein A affinity chromatography was used as feed material. Anion exchange experiment conducted using 5 CV, 20 mM Tris, pH 7.50±0.20 as an equilibration buffer, 5 CV, 20 mM Tris, pH 7.50±0.20 as a wash buffer, 5 CV, 20 mM Tris containing 1 M NaCl, pH 7.50±0.20 as an elution buffer and 4 CV, 0.5 M NaOH was used for cleaning of resin. Elution was performed using step salt gradient. All the process steps were performed at six-minute residence time. Also, anion exchange chromatography was performed using Q Sepharose® Fast Flow resin at pH 7.00±0.20 is shown in FIG. 10. HCP and HCDNA removal by anion exchange chromatography are as shown in table 5.

TABLE 5
Impurity content in anion exchange chromatography output.

| | Mass | Re- | | | HC-DNA | |
|---|---|---|---|---|---|---|
| | balance | covery | HCDNA | HCP | log | HCP log |
| Process step | (%) | (%) | (ng/mL) | (ppm) | reduction | reduction |
| Anion exchange | 100.31 | 95.87 | 54.05 | 16.31 | 0.86 | 0.94 |
| chromatography | | | | | | |
| output | | | | | | |

### Example 6: Cation Exchange Chromatography for Separation of Various Product Related Impurities

Cation exchange chromatography was performed using Fractogel® SO3-(M), POROS® XS, and Capto™ SP ImpRes resin. The flowthrough monoclonal antibody solution of anion exchange chromatography was used as feed material. Cation exchange experiment conducted using 5 CV, 20 mM Sodium acetate, pH 5.00±0.20 as an equilibration buffer, 3 CV, 20 mM Sodium acetate, pH 5.00±0.20 as a wash buffer, 15 CV, 20 mM Sodium acetate containing 1 M NaCl, pH 5.00±0.20 as an elution buffer. Elution was performed using a linear salt gradient from 10% to 100% of elution buffer. All the process steps were performed at five minute residence time. Also, cation exchange chromatography was performed using Fractogel® SO3-(M) at pH 7.00±0.20. HCP and HCDNA removal by cation exchange chromatography are as shown in FIG. 11 and table 6.

TABLE 6
Impurity content in anion exchange and cation exchange
chromatography output

| | | | | | HC- | |
|---|---|---|---|---|---|---|
| | Mass | | | | DNA | HCP |
| | bal- | Re- | | | log | log |
| | ance | covery | HCDNA | HCP | re- | re- |
| Process step | (%) | (%) | (ng/ml) | (ppm) | duction | duction |
| Cation exchange | 98.55 | 76.44 | 3.03 | Below | 1.25 | Not |
| chromatography | | | | detection | | appli- |
| output | | | | limit | | cable |

### Example 7: Analytical Characterization of Monoclonal Antibody Using Various Analytical Techniques

i. Protein A HPLC Analysis for Quantification of Monoclonal Antibody Product

Concentration of monoclonal antibody product in various chromatography outputs was determined using 2.1 mm×30 mm of particle size 20 μm POROS™ A 20 column on Agilent 1200 HPLC system. The mobile phase consisted of 1×PBS pH 7.2±0.20 (Buffer A) and 3% GAA pH 2.5±0.20 (Buffer B). Flow rate was maintained at 1 mL/min using a gradient of A to B for 5 minutes method at a wavelength of 280 nm Quantitative estimation of the monoclonal antibody product in various process outputs was determined using this analytical method, further showed in FIG. 12.

ii. SEC-HPLC the Aggregate Analysis of Monoclonal Antibody Product

Aggregation and fragments in monoclonal antibody products were determined using a 7.8 mm×300 mm YARRA™ SEC 3000 column of particle size 3 nm on Agilent 1200 HPLC system. The mobile phase consisted of sodium phosphate buffer pH 6.5 10±0.20 (Buffer A). The flow rate was maintained at 0.75 mL/min using an isocratic method at a wavelength of 280 nm High molecular weight impurities (HMW), the main peak, and low molecular weight impurities (LMW) of purified monoclonal antibody products were determined using this method, further showed in FIG. 13.

iii. CEX HPLC Analysis for Charge Variant Analysis of Output Monoclonal Antibody Sample:

Acidic and basic variants in monoclonal antibodies were obtained with MAbPac™ SCX-10 column of pore particle 5 nm. The mobile phase consisted of sodium phosphate buffer pH 6 (Buffer A) and sodium phosphate buffer with NaCl pH 6 (Buffer B). Also, charge variants analysis was carried out using YMC Biopro size 5 micron, 4.6*250 mm column on Agilent 1200 HPLC system, mobile phase 20 mM sodium acetate (Buffer A), and 20 mM sodium acetate+300 mM NaCl (Buffer B). The flow rate was maintained at 0.5 mL/min using a gradient method at a wavelength of 220 nm Acidic, basic variants and the main peak of purified monoclonal antibody compared with innovator monoclonal antibody, further showed in FIG. 14.

iv. Intact Mass Analysis of Purified Monoclonal Antibody Using MALDI-TOF Analysis

Intact mass analysis of purified monoclonal antibody product was performed using MALD-TOF analysis. Innovator and purified monoclonal antibodies were mixed in a 1:1 ratio with sinapinic acid to perform MALDI-TOF analysis. Matrix sinapinic acid (20 mg/ml) was prepared in ACN: purified water: Trifluoroacetic acid (TFA) (50:50:0.1). 1 μL of a homogenized mixture of sample and matrix was deposited on a clean 384 well MALDI plate. The plate was inserted into AB SCIEX TOF/TOF™ 5800 instrument. The instrument was used in positive ion linear mode. Nitrogen laser at 337 nm radiation was kept as an ionization source. The MALDI-TOF range was 10 KDa to 400 KDa and laser intensity in between 5000 to 6000 was used for the analysis of samples depicted in FIG. 15. BSA was used as a positive control. Result analysis was performed using Data Explorer software.

v. CD Spectroscopy Analysis

JASCO (J-815) was used for CD Spectroscopy analysis of protein samples. All the samples were analyzed at 25° C. temperature. Data pitch was set to 0.025 nm and 1.00 nm with continuous scanning mode using 1 mm cuvette for Far UV CD and data pitch was set to 1.00 nm with continuous scanning mode using 1 cm cuvette for Near UV CD. Sample used for analysis was 0.2 mg/mL for Far UV CD and 1.5 mg/mL for Near UV CD. The far UV CD spectra scan was between 190 and 240 nm that contains information of the secondary structure of proteins. The Far UV CD spectra of innovator and biosimilar products indicated similar secondary structures. The Near UV CD spectra scan was between 240 and 350 nm contains information of the tertiary structure of proteins. The Near UV CD spectra of innovator and biosimilar products indicated similar structures. FIG. 16 depicts the far CD spectroscopic analysis of the monoclonal antibody.

vi. ELISA Analysis for Host Cell Protein (HCP) Quantitation of Monoclonal Antibody

Host Cell Protein analysis of process samples was performed using a two-site Immuno-enzymatic assay (Cygnus CHO HCP 3rd generation kit F550). Samples containing CHO HCPs were reacted simultaneously with horseradish peroxidase (HRP) enzyme-labeled anti-CHO antibody (goat polyclonal) in microtiter strips coated with an affinity-purified capture anti—CHO antibody. The immunological reactions result in the formation of a sandwich complex of solid-phase antibody—HCP—Enzyme labeled antibody. The microtiter strips are washed to remove any unbound reactants. The substrate, tetramethylbenzidine (TMB) is then reacted. The amount of hydrolyzed substrate is read on a microtiter plate reader at 450 and 650 nm and is directly proportional to the concentration of CHO HCPs present.

vii. ELISA Analysis for Protein a Leach Quantitation of Monoclonal Antibody

The Mix-N-Go Protein A assay is two sites immune enzymatic assay (Cygnus Mix-N-Go Protein A Assay, F 600, F 610). Samples containing protein A were first diluted in the Mix-N-Go sample diluent provided with the kit. The Mix-N-Go denaturing buffer was then added and mixed to dissolute the protein A from the product antibody. The samples are then reacted in microtiter strips coated with a polyclonal anti-protein A capture antibody. A second anti-protein A antibody labeled directly with Horse Radish Peroxidase (HRP) enzyme is simultaneously reacted forming a sandwich complex of solid-phase antibody-protein A: HRP labeled antibody. After a wash step to remove any unbound reactants, the strips are then reacted with tetramethylbenzidine (TMB) substrate. The amount of hydrolyzed substrate is read on a microtiter plate reader at 450 and 650 nm and will be directly proportional to the concentration of protein A present in the sample.

viii. Host Cell DNA Analysis by Using Picogreen Assay

The presence of DNA in various process samples was estimated using Quant-iT™ PicoGreen ds DNA reagent and Kit (Invitrogen, Catalog number: P11496). A standard curve was prepared using double-stranded lambda DNA by diluting 100 μg/mL to 2 μg/mL. The analysis of the process sample was done using 0.1 mL of sample and 0.1 mL of Pico green reagent (used 200 fold diluted reagent). The reaction mixture was incubated for 5 min and fluorescence was measured using fluorescence spectrophotometer, at 480 and 520 nm wavelength (excitation wavelength 480 nm and emission wavelength 520 nm).

### Example 8: Continuous Protein a Capture Step Affinity Chromatography Using the Chromatography Columns

Protein A affinity chromatography was performed using MabSelect™ PrismA resin. Affinity chromatography was performed using three column chromatography system using BioSC™ from Novasep. The concentrated monoclonal antibody solution (SPTFF/ILC output) was used as feed material. Experiment to continuous Protein A affinity chromatography IgG from concentrated material was conducted using 20 mM sodium phosphate containing 150 mM NaCl, pH 7.40±0.20 as an equilibration buffer, 20 mM sodium phosphate containing 500 mM NaCl, pH 7.40±0.20 as a wash buffer, 100 mM glycine-HCl, pH 3.00±0.20 as an elution buffer, 100 mM glycine HCl, pH 2.00±0.20 as a strip buffer and 0.1 M NaOH was used for cleaning of resin. Elution was performed using step pH gradient. All the process steps were performed at two minute residence time. To enhance the process productivity and utilization of Protein A affinity resin, capacity loading at various overall breakthrough percent was performed. For the three column continuous affinity process the productivity of continuous Protein A capture step increases with increase in feed titer as shown in the table below. Table 7 shows the increase in productivity with increase in feed concentration for three column continuous purification process.

TABLE 7
Effect of feed concentration on the productivity of continuous
three column Protein A capture step purification.

| Feed | |
|---|---|
| concentration (g/L) | Productivity (g/L/h) |
| 1 | 14.2 |
| 10 | 21.5 |

### Advantages of the Invention

Use of continuous downstream process platform as in the present invention facilitates reduced consumption of buffer.
Almost five-fold improvements in productivity over the existing manufacturing process were obtained by integrating the continuous chromatographic purification steps for IgG downstream processing.
The resultant IgG's or monoclonal antibodies purified by the present process have high purity in keeping with the innovator product's purity standards.
The IgGs and monoclonal antibodies produced by the present invention retain their therapeutic activity.

We claim:

1. A process for purifying a monoclonal antibody, comprising the steps of:
(a) filtering a CHO harvest material containing the monoclonal antibody using an AEX hybrid filter;
(b) concentrating the AEX hybrid filter output using a first single-pass tangential flow filtration (SPTFF) system or a first in-line concentrator (ILC) to obtain a concentrated monoclonal antibody solution;
(c) subjecting the concentrated monoclonal antibody solution from step (b) to a Protein A affinity chromatography comprising;
i. loading the concentrated monoclonal antibody solution of step (b) at pH range from 6.00 to 9.50 with conductivity more than 1 mS/cm to an affinity chromatography resin to capture monoclonal antibody and remove process and product related impurities,
ii. eluting the captured monoclonal antibody using an elution buffer in pH range from 2.00 to 5.00 with conductivity more than 0.5 mS/cm,
wherein loading on the affinity chromatography resin in step (i) is performed at >0.1% of breakthrough capacity,
wherein number of chromatography columns used in step (i) of affinity chromatography purification is at least one,
wherein elution in affinity chromatography is performed using pH range from 2.00 to 5.00 or a salt based gradient;
(d) subjecting a protein A elution from step (c) for viral inactivation at pH range from 2.00 to 4.00 with a conductivity of more than 0.5 mS/cm;
(e) subjecting the post-viral inactivated liquid output obtained in step (d) to an ion exchange chromatography;
(f) concentrating the ion exchange chromatography output of step (e) using a second single-pass tangential flow filtration (SPTFF II) system or a second in-line concentrator (ILC II) to form a retentate; and
(g) subjecting the retentate of step (f) for a viral filtration to produce the purified monoclonal antibody.

2. The process as claimed in claim 1, wherein the process comprises a batch, an integrated continuous or a pseudo-continuous process.

3. The process as claimed in claim 2, wherein the process is a continuous process.

4. The process as claimed in claim 1, wherein number of the AEX-hybrid filtration units is ≥1.

5. The process as claimed in claim 4, wherein when the AEX-hybrid filtration unit is more than 1, they are connected in-parallel or in-series with a suitable pressure or time or volume or protein concentration based flow and flow path controller to facilitate the AEX hybrid filtration operation in a continuous mode.

6. The process as claimed in claim 1, wherein the AEX-filtration step is performed at a pH ranging from 6.00 to 9.50, with conductivity more than 0.5 mS/cm to an affinity chromatography.

7. The process as claimed in claim 1, wherein number of the single-pass tangential flow filtration (SPTFF) and in-line concentrator (ILC) units is >1.

8. The process as claimed in claim 7, wherein when the single pass tangential flow filtration (SPTFF) or in-line concentrator (ILC) are more than 1, they are connected in-parallel or in-series with a suitable pressure or time or volume or protein concentration based flow and flow path controller to facilitate the SPTFF/ILC operation in a continuous mode.

9. The process as claimed in claim 1, wherein the single-pass tangential flow filtration (SPTFF) and in-line concentrator (ILC) are performed at a pH ranging from 6.00 to 9.50, with conductivity more than 0.5 mS/cm to the affinity chromatography.

10. The process as claimed in claim 1, wherein the AEX-hybrid filtration, and SPTFF and/or ILC units are used in single or multiple times.

11. The process as claimed in claim 1, wherein a feed inlet of both the SPTFF and/or ILC system are connected to an outlet of the AEX hybrid filter.

12. The process as claimed in claim 1, wherein a retentate outlet of both the SPTFF and/or ILC system is connected to the protein A affinity chromatography.

13. The process as claimed in claim 12, wherein the protein A based affinity chromatography is performed using a Protein A ligand immobilized on a chromatography matrix.

14. The process as claimed in claim 13, wherein the Protein A based affinity chromatography is performed using the Protein A ligand immobilized on natural polymer bead based or a Synthetic polymer bead based, a membrane based, a hydrogel based or a fibre based chromatography matrix.

15. The process as claimed in claim 14, wherein the chromatography matrix is selected from the group consisting of MabSelect SuRe™, MabSelect SuRe™ LX, MabSelect SuRe™ pcc, MabSelect™ PrismA, Fibro PrismA, Praesto® Jetted A50, Amsphere™ A3, TOYOPEARL®, AF-rProtein A HC-650F, KANEKA KanCapA™, KANEKA KanCapA™ 3G, Eshmuno® A, Praesto® AP, and MabSpeed™ rP202.

16. The process as claimed in claim 1, wherein the ion-exchange chromatography is selected from the group consisting of an anion exchange and a cation exchange chromatography.

17. The process as claimed in claim 16, wherein the anion exchange chromatography comprises a resin or a membrane, selected from the group consisting of DEAE Sepharose® Fast Flow, Q Sepharose® Fast Flow, SOURCE™ 15Q, SOURCE™ 30Q, Fractogel® EMD DEAE, POROS® 50 HQ, Nuvia™ Q, Capto™ ImpRes Q. Capto™ Q, Capto™ DEAE, Fractogel® EMD TMAE, Fractogel® EMD DMAE, Natrix® Q, Sartobind® Q, and Mustang® Q.

18. The process as claimed in claim 16, wherein the anion exchange chromatography is performed in a flow-through mode for separation of process related impurities at pH 6.50 to 8.50 with conductivity more than 0.5 mS/cm.

19. The process as claimed in claim 18, wherein the anion exchange chromatography is performed using an anion exchange functional group selected from a group consisting of diethylaminoethyl, quaternary ammonium, polyethyleneimine, trimethylammonium ethyl, linked to a bead-based, a membrane-based, a hydrogel-based, and a fiber-based chromatography matrix.

20. The process as claimed in claim 16, wherein the cation exchange chromatography comprises a resin or a membrane selected from the group consisting of Fractogel® EMD SO3-(M), Capto™ SP ImpRes, POROS® XS, Nuvia™ S, SOURCE™ 15S, SOURCE™ 30S, SP Sepharose® Fast Flow, Natrix® HD-Sb, Sartobind® S, and Mustang® S.

21. The process as claimed in claim 20, wherein the cation exchange chromatography is performed in bind and elute mode for separation of product related impurities at pH to 4.00 to 7.00 with conductivity more than 0.5 mS/cm.

22. The process as claimed in claim 21, wherein the cation exchange chromatography is performed using a cation exchange functional group selected from the group consisting of sulfonate group, sulfopropyl group, and sulphonic acid linked to a bead-based, a membrane-based, a hydrogel-based and a fiber based chromatography matrix.

23. The process as claimed in claim 1, wherein the Protein A based affinity chromatography, and ion exchange chromatography are performed using an axial or a radial flow chromatography column.

24. The process as claimed in claim 23, wherein the radial flow chromatography columns are selected from an axial or a radial flow chromatography column.

25. The process as claimed in claim 1, wherein residence time for Protein A based affinity chromatography, anion exchange chromatography and cation exchange chromatography is more than 15 seconds.

26. The process as claimed in claim 3, wherein the continuous process exhibits a volumetric concentration factor (VCF) value of at least 1.1× for different concentrations of monoclonal antibody; wherein, the X is the initial concentration of feed.

27. The process as claimed in claim 3, wherein the process produces a monoclonal antibody yield of 25.59 g/L/h.

28. The process as claimed in claim 3, wherein the purified monoclonal antibody contains no more than 0.3% soluble aggregate.

29. The process as claimed in claim 3, wherein the purified monoclonal antibody contains no more than 100 ppm HCP.

30. The process as claimed in claim 3, wherein the purified monoclonal antibody contains no more than 10 ng/mL of DNA.

31. The process as claimed in claim 3, wherein the monoclonal antibody is an IgG.
